(19) United States
(12) Patent Application Publication
(10) Pub. No.: US 2026/0115233 A1
(43) Pub. Date: Apr. 30, 2026

(54) APPLICATION OF AKKERMANSIA MUCINIPHILA IN AMELIORATION AND TREATMENT OF CEREBRAL MALARIA CAUSED BY PLASMODIUM BERGHEI

(71) Applicants: Hubei University of Medicine, Shiyan City (CN); Tongji University, Shanghai (CN)

(72) Inventors: Jian Li, Shiyan City (CN); Qingfeng Zhang, Shanghai (CN); Junming Tang, Shiyan City (CN); Daiqian Zhu, Jiangshan City (CN); Jinyu Mo, Shiyan City (CN)

(73) Assignees: Hubei University of Medicine, Shiyan City (CN); Tongji University, Shanghai (CN)

(21) Appl. No.: 19/338,063

(22) Filed: Sep. 24, 2025

(30) Foreign Application Priority Data
Oct. 30, 2024 (CN) 202411528863.6

(51) Int. Cl. A61K 35/741; A61K 31/357; A61K 31/40; A61K 31/436; A61K 35/00; A61P 33/06; C12N 1/20

(52) U.S. Cl. CPC A61K 35/741; A61K 31/357; A61K 31/40; A61K 31/436; A61P 33/06; C12N 1/20; A61K 2035/115

(57) ABSTRACT

The disclosure belongs to the technical field of biomedicine, and specifically relates to an application of Akkermansia muciniphila (AKK) in the amelioration and treatment of cerebral malaria caused by Plasmodium berghei. The disclosure aims to evaluate the efficacy of AKK alone or in combination with adjuvant drugs (dihydroartemisinin, rapamycin, and atorvastatin) in ameliorating and treating experimental cerebral malaria in an experimental cerebral malaria model. The results indicate that AKK, whether used alone or in combination with drugs, can effectively ameliorate behavioral deficits and immunopathological damage of each organ, lower parasitemia levels, and prolong survival period of mice. Compared with the use of drug alone, the combination therapy can effectively treat the experimental cerebral malaria. The disclosure is the first to propose the concept of bacterial-drug synergy concept, and provides a solution to the treatment of human cerebral malaria on the basis of the concept.

### TECHNICAL FIELD

The disclosure belongs to the technical field of biomedicine, and specifically relates to an application of Akkermansia muciniphila (AKK) in the amelioration and treatment of cerebral malaria (CM) caused by Plasmodium berghei.

### BACKGROUND

Malaria remains one of the most severe infectious diseases worldwide. In 2021, the World Health Organization (WHO) report showed that the number of malaria cases increased to 247 million, with 619,000 deaths, a slight decline compared to 2020. CM caused by Plasmodium falciparum is a lethal complication of malaria, which is the most common and severe parasitic disease affecting the human central nervous system. Artemisinin (ART) and its derivatives are currently the first-line antimalarial drugs for CM. However, with the long-term use of the antimalarial drugs, the resistance to antimalarial drugs is constantly increasing under the drug pressure. In addition, the antimalarial drugs mainly used to clear Plasmodium from the body, so the use of antimalarial drugs alone fails to comprehensively and effectively ameliorate neurological and cerebral pathological symptoms caused by immunopathological damage. Therefore, it is needed to explore new treatment regimens to assist in drug treatment, and make up for the deficiency of antimalarial drugs, thereby more comprehensively treating CM. Currently, the experimental cerebral malaria (ECM) infected by Plasmodium berghei is the most common used animal model for studying human CM.

AKK is a normal bacterium in the human intestinal tract, and is a mucin-decomposing bacterium. The strain MucT was isolated by Derrien in 2004, which is an oval-shaped and Gram-negative anaerobic bacterium, and is a representative of Verrucomicrobia. AKK is negatively correlated with obesity, diabetes, cardiovascular diseases, and low-grade inflammation. Oral administration of AKK can ameliorate the related symptoms of metabolic diseases in mice. Accordingly, AKK is expected to be a candidate drug for treating type 2 diabetes and obesity. After interventions with AKK, a variety of diseases such as obesity, amyotrophic lateral sclerosis, inflammatory bowel disease and colorectal cancer can be prevented and treated.

However, there are no current reports on AKK in ameliorating or treating CM.

Drug treatment remains the primary method for CM at present. However, decades of use of monotherapy have resulted in resistance to several recommended antimalarial drugs. ART and its derivatives are the first-line antimalarial drugs for treating CM in adults, children, and pregnant women, but the use of antimalarial drugs alone fails to comprehensively ameliorate neural and cerebral pathological damage, and the effectiveness of the antimalarial drugs is reduced. Lacking adjuvant therapies, the mortality rate caused by CM continue to rise. Therefore, inspired by combination therapy concept, there is an urgent need to explore a combination therapy that has a superimposed effect with ART to mitigate or prevent CM. In the disclosure, preliminary studies show that a combination strategy using dihydroartemisinin (DHA) with rapamycin and atorvastatin significantly prolongs survival periods of mice and alleviates immunopathological damage compared with the use of DHA alone. However, the underlying mechanisms remain unclear. Additionally, in early studies, there is significant changes in intestinal flora of mice after being infected with different Plasmodium species, suggesting that the intestinal flora may play a key role. Subsequently, on the basis of triple-drug combination therapy, AKK in the intestinal flora of mice in the triple-drug combination group is significantly increased compared with the use of DHA alone, implying the critical role of AKK in this process.

Studies have shown that AKK plays an essential role in intestinal inflammatory diseases, tumors, neurological disorders, etc. However, there is no report on its role in infectious diseases, particularly in CM. For this purpose, the disclosure aims to elucidate the therapeutic role and underlying mechanisms of AKK in the treatment of ECM. Specifically, an ECM model is first established, subsequently, AKK alone or in combination with other drugs is used to treat on this model, and the therapeutic efficacy of AKK on ECM is observed using behavioral, pathological, and microbiological technical means.

### SUMMARY

The disclosure first provides an application of AKK in the amelioration and treatment of CM caused by Plasmodium berghei. The application is in vivo or in vitro. Optionally, the amelioration and treatment include one or more of the following:
A) ameliorating behavioral performance of mice;
B) ameliorating immunopathological damage of each organ;
C) reducing parasitemia; and
D) prolonging survival periods of the mice.

In some embodiments, the in vitro is a bacterium.

In some embodiments, the in vivo is a mouse.

In some embodiments, the AKK is Akkermansia muciniphila ATCC BAA-835.

The disclosure further provides an application of AKK in the preparation of a drug for ameliorating and treating CM caused by Plasmodium berghei.

In some embodiments, the drug further contains one or three of DHA, rapamycin, and atorvastatin; and the DHA is a required drug.

In some embodiments, the drug further contains DHA.

In some embodiments, the drug further contains DHA and rapamycin.

In some embodiments, the drug further contains DHA, rapamycin, and atorvastatin.

Compared with the prior art, the disclosure at least has the following beneficial effects.

Preliminary results show significant enrichment of AKK in the triple-drug combination group, suggesting that AKK may play a role in the treatment of ECM. The disclosure aims to evaluate the efficacy of AKK alone or in combination with adjunctive drugs (DHA, rapamycin, and atorvastatin) in an ECM model. The results show both the use of AKK alone and in combination with drugs can effectively ameliorate behavioral deficits of mice and immunopathological damage in each organ, reduce parasitemia, and prolong survival periods of mice. Compared with the use of drug alone, the combination therapy can effectively treat ECM. Since all three drugs (DHA, rapamycin, and atorvastatin) are clinical drugs, the translational application time can be shortened if they are applied to treat human CM clinically. The disclosure is the first to propose the concept of bacterial-drug synergy concept, and provides a solution to the treatment of human cerebral malaria on the basis of the concept.

### BRIEF DESCRIPTION OF THE DRAWINGS

FIG. 1A shows morphological observation of AKK under an optical microscope; and

FIG. 1B shows morphological observation of AKK under a scanning electron microscope.

FIG. 2A shows three-view observations of normal mice; and

FIG. 2B shows three-view observations of ECM mice.

FIG. 3A shows effects of different drug treatments on the body weight of ECM mice;

FIG. 3B shows the effects of different drug treatments on rapid murine coma and behavioral scale (RMCBS) of ECM mice;

FIG. 3C shows effects of different drug treatments on survival rate of ECM mice; and

FIG. 3D shows effects of different drug treatment on parasitemia of ECM mice.

FIG. 4A shows a frontal view of brain tissues after different drug treatments;

FIG. 4B shows vascular leakage assessed by Evans Blue (EB) assay; and

FIG. 4C shows intestinal permeability.

FIG. 5A shows brain tissues (1000×) stained with hematoxylin and eosin (HE);

FIG. 5B shows liver tissues (400×) stained with HE;

FIG. 5C shows spleen tissues (400×) stained with HE; and

FIG. 5D shows small intestine tissues (400×) stained with HE.

### DETAILED DESCRIPTION

For clearer technical problems to be solved by the disclosure, technical solutions and advantages, detailed description will be provided by reference to the accompanying drawings and specific embodiments.

### ABBREVIATED TERMS

DHA: dihydroartemisinin;
RAP: rapamycin;
AVA: atorvastatin.

### Embodiment 1 Material and Method

Experimental animal, Plasmodium strain and bacterial strain: female C57BL/6 mice (weighing 18-22 g, 8-10 weeks old) were purchased from HNSJA Co., Ltd. in Changsha, China. The mice were raised under the conditions of no specific pathogen and fed with ultraviolet-irradiated feed and purified water to maintain the experimental condition (25±3° C.) with appropriate living and feeding standards. All mice underwent environmental adaptation one week prior to the experiment.

Plasmodium berghei ANKA (PbA) was passaged and cryopreserved in liquid nitrogen for long-term preservation by our laboratory. Akkermansia muciniphila ATCC BAA-835 was purchased from ATCC (http://www.bncc.org.cn/pro/p75/p_323275.html).

The experimental instruments are shown in Table 1.

TABLE 1
Experimental instruments

| | | Production place and |
|---|---|---|
| Name | Model | company |
| Weighing scale | 76175 | Beiximan, China |
| OLYMMPUS microscope | BX53 | Olympus corporation, |
| | | Japan |
| Centrifugal machine | 5424R | Eppendorf, Germany |
| Constant-temperature | MK-20 | Allsheng, China |
| metal bath | | |
| Tissue section image | BX53 | Olympus corporation, |
| imaging system | | Japan |
| Micropipette | 0.1-2.5 μL, 3123000217 | Eppendorf, Germany |
| | 0.5-10 μL, 3123000225 | |
| | 2-20 μL, 3123000233 | |
| | 10-100 μL, 3123000241 | |
| | 20-200 μL, 3123000250 | |
| | 100-1000 μL, 3123000268 | |
| Multimode reader | SYNERGY-HT | BioTek, US |
| Mouse tail vein injection | KW-XXY | Calvin, China |
| instrument | | |
| Fridge | −20° C., −80° C. | Haier, China |

The experimental reagents and consumables are shown in Table 2.

TABLE 2
Experimental reagents and kits

| Name | Manufacturer |
|---|---|
| Giemsa dye liquor | Yulu, China |
| DHA | Meryer, China |
| Rapamycin | Meryer, China |
| Atorvastatin | Solarbio, China |
| Syringe | Weigao, China |
| Frozen pipe | Thermo Fisher Scientific, US |
| Glass slide | Citotest, China |
| Methanol | Zhongtian, China |
| Dimethylsulfoxide (DMSO) | VWR, America |
| EB | SIGMA, Germany |
| Formamide | SIGMA, Germany |
| 0.9% normal saline | Kelun, China |
| General-purpose tissue fixative | Absin, China |
| Slide storage box | Beyotime, China |
| PBS buffer powder | Servicebio, UK |
| Pipette tip | Thermo Fisher Scientific, US |

### Embodiment 2

Resuscitation and passage: the PbA cryopreserved in liquid nitrogen was placed in a 37° C. water bath; after thawing, the blood was aspirated using a 1 mL syringe and immediately inoculated intraperitoneally into C57BL/6 mice at a dose of 0.2 mL per mouse, and the process was the blood-inoculated resuscitation. When the parasitemia level in the blood-inoculated mice reached 15%-30%, the eyeballs were removed for blood collection, and the blood was placed into a heparin-anticoagulated tube, and was passaged to offspring C57BL/6 mice at a dose of 0.2 mL per mouse according to 1×10⁶ infected red blood cells (iRBCs).

### (1) AKK Bacterial Solution Culture and Extracellular Vesicle Preparation

Culture of AKK: the bacterium was cultured anaerobically at 37° C. in a medium (containing 3.85 g brain heart infusion, 1.6 g soy peptone, 1.13 g anhydrous glucose, 0.55 g N-acetylglucosamine, 0.4 g L-threonine, and 0.05 g L-cysteine per 100 mL); the culture was performed for 3 days until the bacterial solution was slightly turbid; after counting, bacterial pellet was collected by centrifugation, washed, and resuspended in phosphate-buffered saline (PBS) for later use.

### (2) Experimental Grouping and Drug Treatment

60 female C57BL/6 mice were randomly divided into 6 groups: untreated infection

group (PbA), DHA treatment group (DHA), DHA combined with RAP and AVA treatment group (DHA.RAP.AVA), PbA+AKK group, DHA+AKK group and DHA.RAP.AVA+AKK group.

DHA, RAP, and AVA were dissolved in a solution containing 5% DMSO and 0.9% NaCl at doses of 3 mg/kg, 5 mg/kg, and 40 mg/kg, respectively. On the 3rd day post-infection, 5% DMSO was intraperitoneally injected to the PbA group as a control. 3 mg/kg DHA was administered to the DHA group. 5 mg/kg RAP, 3 mg/kg DHA and 40 mg/kg AVA drugs were administered to the DHA.RAP.AVA group. 200 μL of 1×10⁹ cfu/mL AKK bacterial solution was administered to the three groups containing AKK (PbA+AKK group, DHA+AKK group, and DHA.RAP.AVA+AKK group) via oral gavage daily for 14 consecutive days from day 0 post-infection. On the 3rd day post-infection, treatment with 3 mg/kg DHA, 3 mg/kg DHA+5 mg/kg RAP+40 mg/kg AVA was performed for 5 consecutive days at a volume of 200 μL/d.

### (3) Detection of Basic Indicators

Observation of parasitemia: blood was collected from the tail vein to prepare a thin blood smear, and the blood smear was subjected to Giemsa staining. Parasitemia was assessed using an optical microscope (10× eyepiece, 100× objective lens) by examining the Giemsa-stained thin blood smear. The parasitemia was examined and quantified by counting the iRBCs among at least 1,000 red blood cells.

(4) Detection of Basic Behavioral Indicators: From Day 0 Post-Infection, the Body weight, neurological characteristics, parasitemia, and survival rate of the mice were monitored daily. Neurological characteristics were evaluated using RMCBS, and the evaluation was performed using 10 parameters (fur, limb strength, defensive ability, auricle reflex, toe reflex, contact response, gait, balance, body posture, and exploratory activity). If the mice gradually exhibited the characteristics such as unsteady walking, ataxia, messy fur, no stretching, loss of toe reflex, loss of auricle reflex, convulsion, coma, and even death, the ECM model was successfully constructed. Parasitemia was assessed and quantified by examining the thin blood smear under a 100× optical microscope to count the number of iRBCs among 1,000 red blood cells.

(5) Assessment of blood-brain barrier (BBB) integrity: the protective effect on the brain was evaluated by assessing the BBB integrity. 1% EB dye was diluted in 0.9% NaCl. On the 8th day post-infection, the mice were intravenously injected with 200 μL of 1% EB dye liquor via the tail vein, allowing the dye to circulate for 30 minutes. Subsequently, the mice were anesthetized, and 0.9% NaCl was perfused through the heart into the right atrium until the clear liquid flowed out. The brains were rapidly dissected, photographed, and weighed, then placed in 1.5 mL centrifuge tubes for grinding. Each brain sample was added with 1 mL of formamide and incubated in a constant temperature metal bath of 37° C. for 48 hours. The samples were then centrifuged at 1000 rpm for 10 minutes, and the absorbance was measured at 630 nm using a microplate reader. The EB was quantified on the basis of a standard curve.

(6) Histopathological observation: tissue sampling was performed after the completion of drug administration, that is, on the 8th day post-infection. After euthanasia, brain, liver, spleen, and small intestine tissues were immediately collected and washed three times in cold PBS to remove blood. Subsequently, the tissues were fixed using a general-purpose tissue fixative for 24 hours and embedded in paraffin. Serial sections with a thickness of 4 μm were prepared and stained with HE. Microvascular obstruction and leakage were observed. The sections were observed using an optical microscope, and images were captured using Olympus cellSens Standard 1.13 software.

### Results:

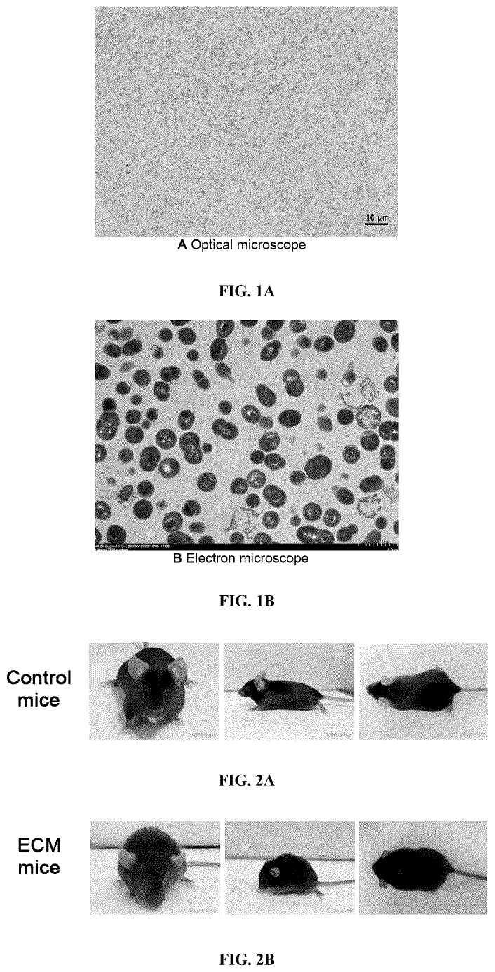

(1) FIGS. 1A-1B show the microscopic morphological images of AKK.

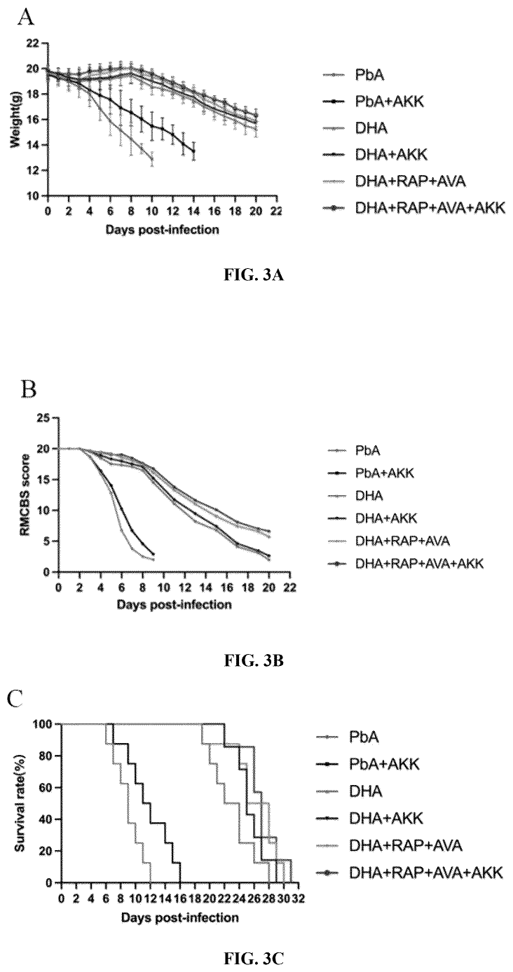

(2) Successful construction of the ECM model: an ECM animal model was constructed by infecting C57BL/6 mice with PbA. C57BL/6 mice were infected with 1×10⁶ iRBCs via intraperitoneal injection, resulting in weight loss, and death starting on days 6-8. RMCBS scoring results show ECM symptoms, and anemia is also observed via tail blood sampling, suggesting that the ECM model is successfully constructed (see FIGS. 2A-2B).

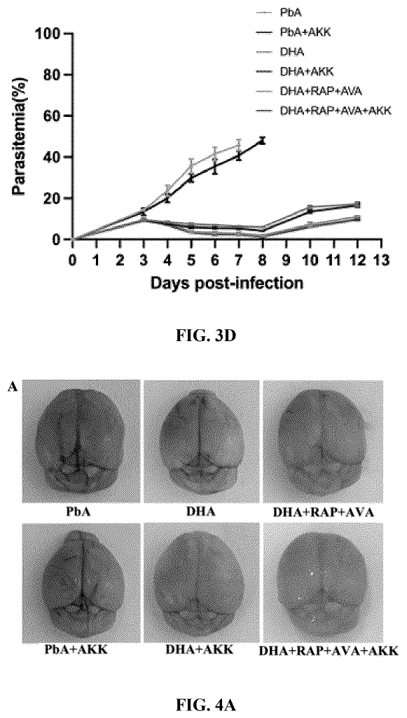

(3) Evaluation of basic indicators: detection of basic behavioral indicators shows that the DHA+RAP+AVA+AKK treatment group has the highest body weight, RMCBS scores, and survival rates (FIGS. 3A-3C), and the lowest parasitemia levels (FIG. 3D), suggesting that AKK may play a synergistic role in combined bacteria-drug therapy for ECM (see Table 3).

TABLE 3
Record of average values for basic indicators

| | | RMCBS | Survival | |
|---|---|---|---|---|
| | Weight | score | rate | Parasitemia |
| Group | (D 14) | (D 9) | (D 16) | (D 8) |
| PbA | — | 2 | 0 | — |
| PbA + AKK | 13.4350 g | 2.9100 | 0 | 48.482% |
| DHA | 17.1273 g | 14.5177 | 100 | 5.855% |
| DHA + AKK | 17.7218 g | 15.2100 | 100 | 4.262% |
| DHA + RAP + AVA | 18.0282 g | 16.3792 | 100 | 1.874% |
| DHA + RAP + | 18.1617 g | 16.7500 | 100 | 1.277% |
| AVA + AKK | | | | |

Note
the time points selected in the table are based on data from the PbA + AKK group as the reference benchmark, where D 8, D 9, D1 4, and D 16 represent days post-infection.

Compared with the PbA+AKK group (AKK intervention alone in ECM model) and the DHA group (DHA treatment alone in ECM model), the DHA+AKK group (combined DHA treatment and AKK intervention in ECM model) has a 31.97% [(DHA+AKK group−PbA+AKK group)/PbA+AKK group] and 3.47% [(DHA+AKK group−DHA group)/DHA group] increase in average body weight of mice, respectively, on day 14; on day 9, the RMCBS scores of mice in the DHA+AKK group improves by 422.68% [(DHA+AKK group−PbA+AKK group)/PbA+AKK group] and 4.77% [(DHA+AKK group−DHA group)/DHA group] compared with that in the PbA+AKK group and DHA group, respectively; and on day 8, parasitemia levels of mice in the DHA+AKK group decreases by 91.21% [(DHA+AKK group−PbA+AKK group)/PbA+AKK group] and 27.21% [(DHA+AKK group−DHA group)/DHA group] compared with that in the PbA+AKK group and DHA group, respectively.

Compared with the PbA+AKK group (AKK intervention alone in ECM model) and the DHA+RAP+AVA group (DHA+RAP+AVA treatment alone in ECM model), the DHA+RAP+AVA+AKK group (combined DHA+RAP+AVA treatment and AKK intervention in ECM model) has a 35.18% [(DHA+RAP+AVA+AKK group−PbA+AKK group)/PbA+AKK group] and 6.04% [(DHA+RAP+AVA+AKK group−DHA+RAP+AVA group)/DHA+RAP+AVA group] increase in average body weight of mice, respectively, on day 14; on day 9, RMCBS scores of mice in the DHA+RAP+AVA+AKK group improves by 475.6% [(DHA+RAP+AVA+AKK group−PbA+AKK group)/PbA+AKK group] and 15.38% [(DHA+RAP+AVA+AKK group−DHA+RAP+AVA group)/DHA+RAP+AVA group] compared with that in the PbA+AKK group and DHA+RAP+AVA group, respectively; and on day 8, parasitemia levels of mice in the DHA+RAP+AVA+AKK group decreases by 97.47% [(DHA+RAP+AVA+AKK group−PbA+AKK group)/PbA+AKK group] and 79.04% [(DHA+RAP+AVA+AKK group−DHA+RAP+AVA group)/DHA+RAP+AVA group] compared with that in the PbA+AKK group and DHA+RAP+AVA group, respectively.

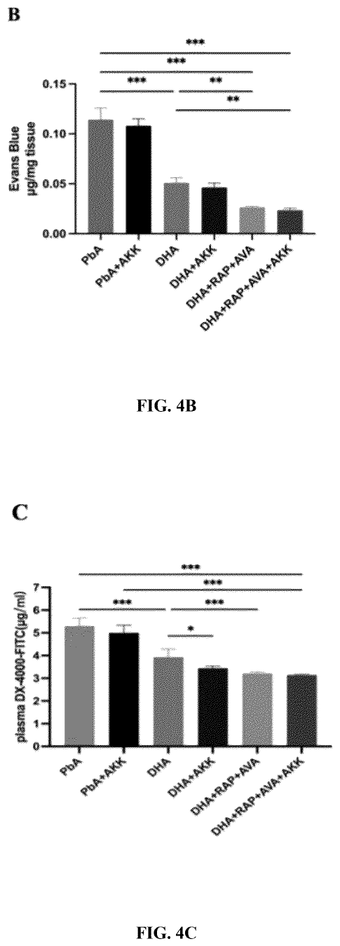

(4) BBB and intestinal permeability: the results of BBB permeability assessment experiment show that brain tissue staining is the lightest in the DHA+RAP+AVA+AKK group (FIG. 4A), with the lowest EB leakage (FIG. 4B). Intestinal permeability experiment shows lower optical density (OD) values in the AKK group (FIG. 4C). These results suggest that AKK-assisted therapy enhances mucosal barrier function of the mice, and both the BBB and intestinal permeability of the mice are improved.

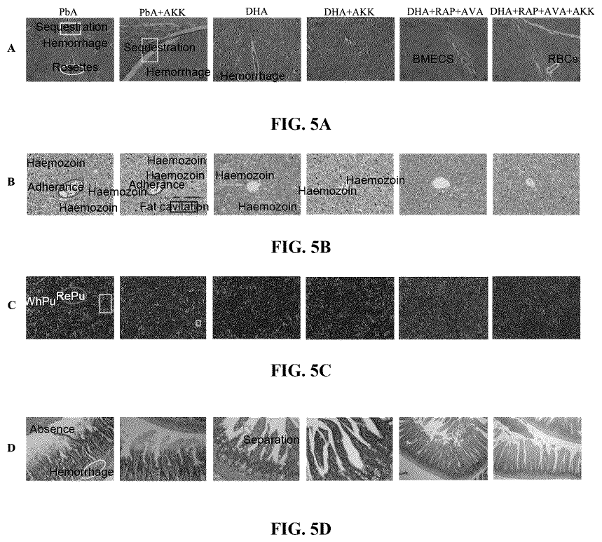

(5) Pathological analysis of visceral organs: FIGS. 5A-5D show histopathological staining of brain, liver, and spleen tissues.

The foregoing is only preferred embodiments of the disclosure, and it is to be noted that those ordinary skilled in the art may make several improvements and embellishments without departing from the principles of the disclosure, and these improvements and embellishments are considered to be within the scope of protection of the disclosure.

What is claimed is:

1. A method of using Akkermansia muciniphila in the preparation of a drug for ameliorating and treating cerebral malaria caused by Plasmodium berghei, comprising the following steps:
anaerobically culturing Akkermansia muciniphila in a culture medium, performing centrifugation to collect a bacterial pellet, washing the bacterial pellet, and resuspending the washed bacterial pellet in a phosphate buffer saline to obtain a bacterial solution of Akkermansia muciniphila, and
administering the bacterial solution of Akkermansia muciniphila to a subject, wherein
the amelioration and treatment comprise one or more of the following:
A) ameliorating behavioral performance of mice,
B) ameliorating immunopathological damage of each organ,
C) reducing parasitemia, and
D) prolonging survival periods of the mice; and
the Akkermansia muciniphila is Akkermansia muciniphila ATCC BAA-835.

2. The method according to claim 1, wherein the drug further contains one or three of dihydroartemisinin, rapamycin, and atorvastatin, the dihydroartemisinin being a required drug.

3. The method according to claim 1, wherein the drug further contains dihydroartemisinin.

4. The method according to claim 1, wherein the drug further contains dihydroartemisinin, rapamycin, and atorvastatin.
